(19) United States
(12) Patent Application Publication
(10) Pub. No.: US 2023/0405203 A1
(43) Pub. Date: Dec. 21, 2023

(54) Method of Dialysis of Blood

(71) Applicant: Medical Components, Inc., Harleysville, PA (US)

(72) Inventors: John William Timko, Rochester, MI (US); Mark Steven Fisher, Sellersville, PA (US)

(21) Appl. No.: 18/240,070

(22) Filed: Aug. 30, 2023

Related U.S. Application Data
(62) Division of application No. 16/819,364, filed on Mar. 16, 2020.
(60) Provisional application No. 62/819,038, filed on Mar. 15, 2019.

(51) Int. Cl. A61M 1/36; A61M 1/16; A61M 1/28; A61M 39/06; A61M 39/10; A61M 39/28

(52) U.S. Cl. CPC A61M 1/3661; A61M 1/1621; A61M 1/285; A61M 39/06; A61M 39/10; A61M 39/28; A61M 2039/062; A61M 2039/0673; A61M 2205/3334; A61M 2025/0031

(57) ABSTRACT

The present disclosure provides a method for hemodialysis of blood, comprising providing a hemodialysis catheter having a low recirculation distal tip, a first and a second extension tubes that are configured to be connected to a dialysis machine, providing a hemodialysis machine with an arterial patient connector and a venous patient connector; conducting hemodialysis with the first extension tube connects to the arterial patient connector of the dialysis machine, and the second extension tube connects to the venous patient connector of the dialysis machine; and periodically reversing the first and second extension tubes connections to the arterial and venous patient connectors of the dialysis machine in a subsequent hemodialysis. The present disclosure also provides a hemodialysis catheter, comprising a switchable connection indicator indicating connection to arterial or venous flow path. The switchable connection indicator can be integrated with a clamp.

### CROSS REFERENCE TO RELATED APPLICATIONS

This application is a divisional application of U.S. patent application Ser. No. 16/819,364, filed Mar. 16, 2020, which claims the benefit of U.S. provisional application No. 62/819,038, filed Mar. 15, 2019, the contents of which applications are incorporated herein by reference.

### FIELD OF THE INVENTION

This invention relates to the dialysis of blood in general, and more particularly to methods and apparatus for use in the same.

### BACKGROUND OF THE INVENTION

Patients with kidney failure routinely require dialysis. Dialysis helps removing waste, salt and extra water to prevent them from building up in the body, keeps a safe level of certain chemicals in your blood, such as potassium, sodium and bicarbonate, and helps controlling blood pressure. In hemodialysis, an artificial kidney (hemodialyzer) is used to remove waste and extra chemicals and fluid from the blood stream. An access (entrance) into the blood vessels is created to get blood into the artificial kidney. This is typically done by minor surgery. A catheter, typically a narrow polymer tube can be inserted into a large vein in the neck. This type of access may be temporary, but is sometimes used for long-term treatment.

Thrombosis and thrombotic occlusion of hemodialysis catheters is a recognized complication that limits hemodialysis blood flow due to reduced catheter patency. Specifically, the tip region in these catheters is significantly affected by thrombosis due to highly disturbed flow patterns at this location. The association between disturbed blood flow and thrombosis has been studied extensively and an important aspect of this association is that the direction of thrombus growth is generally aligned with the flow direction.

Blood flow during a hemodialysis session through hemodialysis catheters is typically unidirectional wherein blood enters the catheter through the uptake lumen (often referred to as the ‘arterial’ lumen) and returns to the body through the return lumen (often referred to as the ‘venous’ lumen). Maintaining this unidirectional flow causes thrombus initiation at the uptake lumen tip and thrombus growth along the flow direction in this region whereby thrombotic occlusion of this location reduces luminal patency and hemodialysis blood flow. When this is observed (usually a few weeks or a few months after catheter implantation in the patient), the direction of blood flow may be reversed during a hemodialysis session to recover some blood flow and extend the patency of the catheter. Such flow reversal approaches are particularly facilitated by hemodialysis catheters that feature a symmetric tip design such as the Symetrex™ hemodialysis catheter.

It should be noted that this type of flow reversal is currently a reactive strategy. A more proactive approach may be contemplated wherein the direction of blood flow is regularly and deliberately altered/reversed (e.g. weekly) without waiting for a thrombotic occlusion event to force the reversal in flow direction. This proactive approach is predicated on disrupting the initiation and growth of a thrombus by regularly changing the direction of blood flow and not permitting stabilization of the thrombus along a direction.

Thus, a study was designed to test this premise and to compare the impact of alternating intraluminal blood flow direction on hemodialysis catheter thrombosis and thrombotic occlusion with conventional unidirectional blood flow.

### SUMMARY OF THE INVENTION

The present disclosure provides a method for hemodialysis of blood, comprising providing a hemodialysis catheter having a first and a second extension tubes, a body comprising at least a first and second lumen, and a low recirculation distal tip, wherein the first and second extension tubes are configured to be connected to a dialysis machine, wherein the first lumen is in fluid communication with the first extension tube, and the second lumen is in fluid communication with the second extension tube, wherein the low recirculation distal tip comprising a first and second distal openings, wherein the first distal opening is in fluid communication with the first lumen and the first extension tube, and the second distal opening is in fluid communication with the second lumen and the second extension tube; providing a hemodialysis machine with an arterial patient connector and a venous patient connector; conducting hemodialysis with the first extension tube connects to the arterial patient connector of the dialysis machine, and the second extension tube connects to the venous patient connector of the dialysis machine; and periodically reversing the first and second extension tubes connections to the arterial and venous patient connectors of the dialysis machine in a subsequent hemodialysis.

In certain embodiments of the disclosure, the hemodialysis catheter may further comprise a hub, a clamp on each of the first and second extension tubes, or a connector on each of the first and second extension tubes.

The method for hemodialysis of blood disclosed herein, may be conducted by periodically reversing the first and second extension tubes connections is by conducting hemodialysis with the first extension tube connects to the arterial patient connector of the dialysis machine, and the second extension tube connects to the venous patient connector of the dialysis machine; and conducting a subsequent hemodialysis with the first extension tube connects to the venous patient connector of the dialysis machine, and the second extension tube connects to the arterial patient connector of the dialysis machine.

The method for hemodialysis of blood disclosed herein, may utilize a low recirculation catheter, wherein the low recirculation distal tip having less than 5% recirculation rate when tested in vitro.

The method for hemodialysis of blood disclosed herein, may utilize a low recirculation catheter, wherein the low recirculation distal tip having less than 2% recirculation rate when tested in vitro.

The method for hemodialysis of blood disclosed herein, may utilize a low recirculation catheter, wherein the low recirculation distal tip having less than 1% recirculation rate when tested in vitro.

The method for hemodialysis of blood disclosed herein, may utilize a low recirculation catheter, wherein the low recirculation distal tip having symmetric first and second distal openings.

The method for hemodialysis of blood disclosed herein, may utilize a low recirculation catheter, wherein the first and second distal openings are symmetric by rotation about a central axis of the catheter body.

The method for hemodialysis of blood disclosed herein, may utilize a low recirculation catheter, wherein the first and second distal openings are symmetric by mirror image about a center line bisecting the catheter body.

The method for hemodialysis of blood disclosed herein, may further comprise indicating on the hemodialysis catheter which extension tube is connected to the arterial patient connector of the dialysis machine.

The method for hemodialysis of blood disclosed herein, may further comprise indicating on the hemodialysis catheter which extension tube is connected to the venous patient connector of the dialysis machine.

The method for hemodialysis of blood disclosed herein, may utilize a low recirculation catheter, wherein the hemodialysis catheter further comprising a connection indicator located at least on one of a hub, extension tube, clamp, and connector.

The method for hemodialysis of blood disclosed herein, may utilize a low recirculation catheter, wherein the connection indicator can be switched from indicating connection to the arterial patient connector to connection to the venous patient connector, and vice versa.

The method for hemodialysis of blood disclosed herein, may further comprise the step of setting the connection indicator to indicate a dialysis machine connection state of a dialysis treatment.

The present disclosure also provides a hemodialysis catheter, comprising a first and a second extension tubes; a body comprising at least a first and second lumen; a low recirculation distal tip; a hub connecting the body and the first and second extension tubes; a clamp on each of the first and second extension tubes; and a connector on each of the first and second extension tubes, wherein the first and second extension tubes are configured to be connected to a dialysis machine, wherein the first lumen is in fluid communication with the first extension tube, and the second lumen is in fluid communication with the second extension tube, wherein the low recirculation distal tip comprising a first and second distal openings, wherein the first distal opening is in fluid communication with the first lumen and the first extension tube, and the second distal opening is in fluid communication with the second lumen and the second extension tube, wherein at least one of the extension tubes, clamps, connectors, comprising a visual or tactile indicator, indicating that the hemodialysis catheter can be used for alternating flow.

In certain embodiments of the hemodialysis catheter as disclosed herein, the at least one of the extension tubes, clamps, connectors for arterial flow path and venous flow path are marked of the same color.

In other embodiments of the hemodialysis catheter as disclosed herein, the at least one of the extension tubes, clamps, connectors for arterial flow path and venous flow path are green or with green marking.

In further embodiments of the hemodialysis catheter as disclosed herein, the at least one of the extension tubes, clamps, connectors for arterial flow path and venous flow path are marked with the same color except red and blue.

In certain embodiments of the hemodialysis catheter as disclosed herein, the at least one of the extension tubes, clamps, connectors for arterial flow path and venous flow path are marked with the same color except red, blue, and green.

The present disclosure further provides a switchable connection indicator for a catheter, comprising a barrel section, and a slidable sleeve, wherein the slidable sleeve can be slid along the barrel section between two positions, wherein a first position of the slidable sleeve on the barrel section indicate arterial connection, and a second position of the slidable sleeve on the barrel section indicate venous connection.

In certain embodiments of the switchable connection indicator disclosed herein, sliding the slidable sleeve on the barrel section exposes a first marking indicating arterial or venous connection, and occluding a second marking indicating arterial or venous connection.

In certain embodiments of the switchable connection indicator disclosed herein, the slidable sleeve is configured to remain at the first or the second position set by a user.

In certain embodiments of the switchable connection indicator disclosed herein, the slidable sleeve and the barrel section comprising corresponding structure for maintaining the slidable sleeve at first or the second position.

In certain embodiments of the switchable connection indicator disclosed herein, the slidable sleeve and the barrel section comprising a depression and a protrusion.

In certain embodiments of the switchable connection indicator disclosed herein, there is a first depression is located on an exterior of the barrel section proximal to one end of the barrel section, and a second depression is located proximal to another end of the barrel section, an a protrusion is located on an interior surface of the slidable sleeve facing the barrel section, which can be accommodated by the first and second depressions on the barrel section, preventing accidental movement of the slidable sleeve.

The present disclosure further provides a clamp configured to be placed along a tube comprising a slide, wherein the slide can be slid along a side of the clamp between two positions, wherein a first position of the slide indicates arterial connection, and a second position of the slide indicate venous connection.

In certain embodiment of the clamp disclosed herein, sliding the slide exposes a first marking indicating arterial or venous connection, and occluding a second marking indicating arterial or venous connection.

In certain embodiment of the clamp disclosed herein, the slide is configured to remain at the first or the second position set by a user.

The present disclosure also provides a hemodialysis catheter, comprising a first and a second extension tubes; a body comprising at least a first and second lumen; a low recirculation distal tip; a hub connecting the body and the first and second extension tubes; a clamp on each of the first and second extension tubes; a connector on each of the first and second extension tubes; and a switchable connection indicator indicating connection to arterial or venous flow path, wherein the first and second extension tubes are configured to be connected to a dialysis machine, wherein the first lumen is in fluid communication with the first extension tube, and the second lumen is in fluid communication with the second extension tube, wherein the low recirculation distal tip comprising a first and second distal openings, wherein the first distal opening is in fluid communication with the first lumen and the first extension tube, and the second distal opening is in fluid communication with the second lumen and the second extension tube.

In certain embodiment of the hemodialysis catheter disclosed herein, the switchable connection indicator is a switchable connection indicator previously described.

In certain embodiment of the hemodialysis catheter disclosed herein, the switchable connection indicator is a clamp previously described.

### BRIEF DESCRIPTION OF THE DRAWINGS

FIG. 1 is a product image of an exemplary symmetric tip hemodialysis catheter;

FIG. 2 shows one exemplary setup of the in-vitro test model. Each experiment consisted of 2 parallel, independent flow systems (Test v Control) circulating blood from the same animal;

FIG. 3 indicates percentage loss of patency showed that the Control group (unidirectional flow) had a greater loss of patency compared with the Test group (alternating flow;

FIGS. 4A & 4B show the thrombus accumulation study showed that thrombus formation in the tip region of the Test group (alternating flow) catheters was significantly lower than the Control group (unidirectional flow) catheters;

FIG. 5. illustrates thrombus accumulation in the tip region of the Test group (alternating flow) catheters was significantly less than the Control group (unidirectional flow) catheters based on radiolabeled platelets-based thrombus quantification;

FIG. 6-8 are embodiments of connection indicators that can be switched between arterial connection (as indicated by the letter A) and venous connection (as indicated by the letter V); and

FIG. 9A-C show an embodiment of a clamp with integrated connection indicators that can be switched between arterial connection (as indicated by the letter A) and venous connection (as indicated by the letter V).

### DETAILED DESCRIPTION

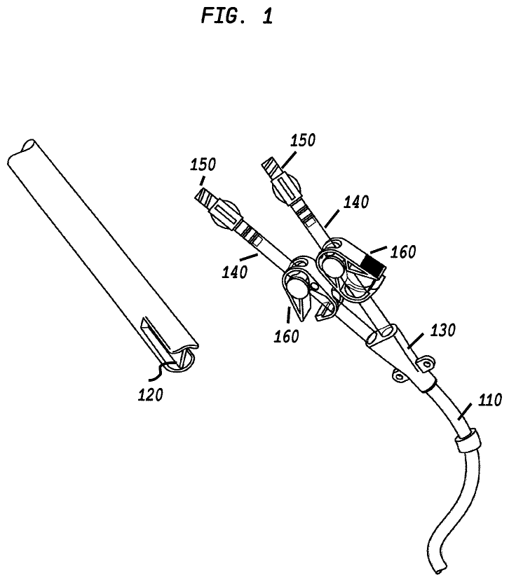

Symetrex™ hemodialysis catheter 100 is a long term hemodialysis catheter marketed by Medical Components, Inc. The Symetrex™ hemodialysis catheter is described in U.S. Pat. No. 10,004,842, the disclosure of which is incorporated herein by reference. FIG. 1 illustrates an exemplary hemodialysis catheter 100. Generally, hemodialysis catheters comprise a catheter body 110, a hub 130, at least two extension tubes 140, and at the distal ends connectors 150 for connecting to hemodialysis machines. The catheter shown in FIG. 1 has a symmetrical distal tip 120. The Symetrex™ hemodialysis catheter has very low recirculation when tested in vitro. The in vitro recirculation rate of Symetrex™ hemodialysis catheter can be lower than 1%. In vitro recirculation rate of can be measured by methods similar to what is described by Vesely, T. M., & Ravenscroft, A. in Hemodialysis Catheter Tip Design: Observations on Fluid Flow and Recirculation. (The Journal of Vascular Access, 17(1), 29-39 (2016). https://doi.org/10.5301/jva.5000463). The method referenced therein is incorporated by reference.

An in-vitro blood flow model was employed to test the hypothesis that alternating intraluminal blood flow direction regularly (Test group) in the Symetrex™ hemodialysis catheter would be associated with less thrombus accumulation in the tip region and less thrombotic occlusion compared with conventional unidirectional blood flow (Control group).

This in-vitro model has been used extensively to study various aspects of hemodialysis catheter thrombosis and thrombosis in numerous other blood contacting medical devices. It represents a controlled system that enables expedited assessments of device thrombosis and is recognized as a suitable model for such assessments by the US FDA and other regulatory agencies¹⁻⁷.

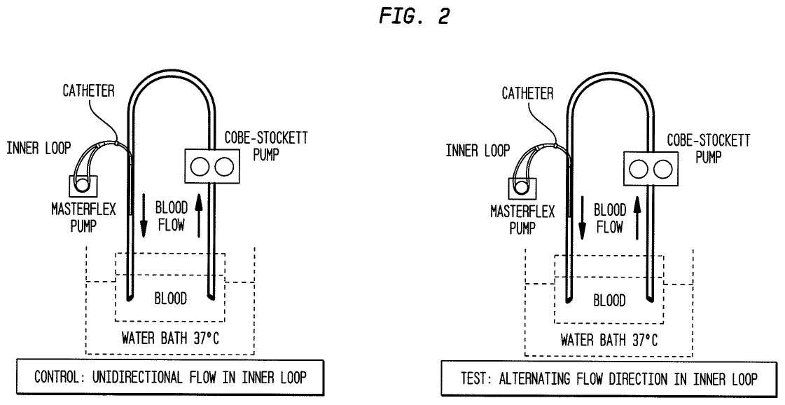

For these studies, fresh heparinized bovine blood (0.5-2 U/ml final heparin concentration) was divided into 2 portions to simultaneously compare the Test and Control. The blood flow circuit consisted of a 12.5 mm ID Tygon tubing outer loop (simulating physiological venous flow) into which the catheter was inserted and sealed and a 6.4 mm ID Tygon tubing inner (‘hemodialysis’) loop (FIG. 2). The blood flow rate in the outer loop was 1.2 L/min (enabled by Cobe-Stockert peristaltic pumps) and the blood flow rate in the inner loop was initially set at 300 ml/min (enabled by Masterflex peristaltic pumps). The flow rates were set and monitored using a Transonic ultrasonic flow probe.

The in-vitro test model is illustrated in FIG. 2. Each experiment consisted of 2 parallel, independent flow systems (Test v Control) circulating blood from the same animal.

Two studies were conducted with this in-vitro model: one study to examine thrombotic occlusion (luminal patency) and another study to examine thrombus accumulation in the tip region.

In the thrombotic occlusion study, the ‘hemodialysis’ blood flow rate in the inner loop was monitored using an ultrasonic flow probe as an indicator of catheter patency and thrombotic occlusion. The flow direction in the Test group was reversed every 2.5 minutes, and the experiment was terminated when the inner loop flow rate in either the Control or Test group dropped by at least 40% from the initial flow rate (of 300 ml/min) due to thrombotic occlusion. The final flow rate as a % of initial flow rate of the Test and Control at termination was compared across N=5 replications using a two-tailed paired t-test.

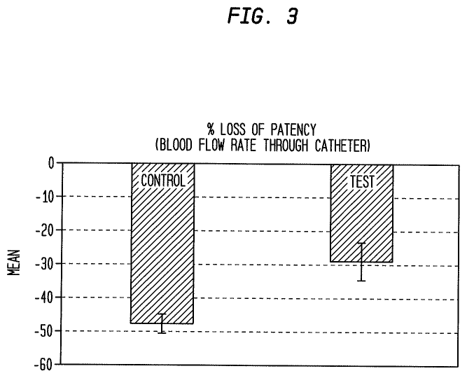

As shown in FIG. 3, the thrombotic occlusion study showed that the Control group (unidirectional blood flow) was associated with a greater decrease in blood flow rate (48%±2%: mean±s.e.m) compared with the Test group with alternating flow direction (29%±6%: mean±s.e.m); Two-tailed paired t-test p-value<0.05, N=5. This finding implied that alternating blood flow direction regularly may help improve catheter patency and hence maintenance of hemodialysis blood flow.

The data graph shown in FIG. 3. Illustrates that the percentage loss of patency [100*(initial flow rate-final flow rate)/initial flow rate)] showed that the Control group (unidirectional flow) had a greater loss of patency compared with the Test group (alternating flow); P<0.05, N=5.

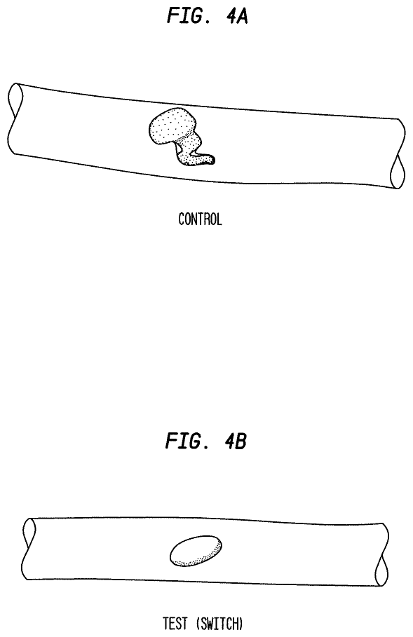

As shown in FIGS. 4A & 4B, the thrombus accumulation study showed that thrombus formation in the tip region of the Test group catheters was significantly (77%+9%: mean±s.e.m) lower than the Control group catheters (Two-tailed paired t-test p-value<0.05, N=5).

Thrombus accumulation in the tip region of the Test group (alternating flow, FIG. 4B) catheter was visibly less than the Control group (unidirectional flow, FIG. 4A) catheter in this representative experiment.

In the thrombus accumulation study, autologous radiolabeled platelets were used to quantify thrombus accumulation at the tip of the Control and Test group catheters⁴⁻⁷. As in the thrombotic occlusion study, the flow direction in the Test group was reversed every 2.5 minutes, and the experiments lasted up to 60 min. The radiation from the radiolabeled platelets in the thrombus in the tip region at the end of experiment in the Test and Control group catheters was compared across N=5 replications using a two-tailed paired t-test.

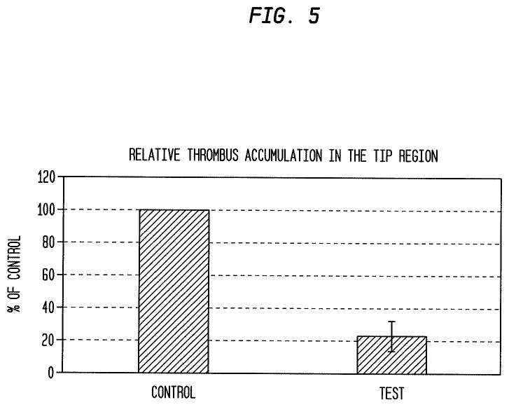

FIG. 5 illustrates results for the thrombus accumulation study. Thrombus accumulation in the tip region of the Test group (alternating flow) catheters was significantly less than the Control group (unidirectional flow) catheters based on radiolabeled platelets-based thrombus quantification (P<0.05, N=5).

The results of these in-vitro studies unexpectedly show that regularly alternating intraluminal blood flow direction in the Symetrex™ hemodialysis catheters is associated with lower thrombus accumulation in the tip region and improved maintenance of patency compared with conventional unidirectional flow.

The in-vitro model used in this study provides a valuable assessment of relative thrombosis characteristics. The conditions (e.g. blood flow, anticoagulation) in this in-vitro model are more controlled than in in-vivo models and the clinical situation, enabling direct comparisons of thrombosis properties between the Test and Control groups. Extraneous parameters such as vessel size, animal activity, variable hemostasis and homeostasis, and infection that can confound in-vivo assessments can be eliminated in the in-vitro model. This allows the evaluation to be focused on a specific variable (alternating vs unidirectional flow in this study), with other parameters remaining relatively constant.

This in-vitro model represents an expedited assessment of relative catheter thrombosis characteristics because the underlying biological process (e.g. platelet activation, adhesion, and aggregation) occur more rapidly in this in-vitro model compared to in-vivo conditions. Past studies that have compared catheters in-vitro and in-vivo have shown that the relative performances are similar in-vitro and in-vivo, and that the thrombus formation which can take days, weeks, or months in-vivo occurs in minutes or hours in-vitro⁴⁻⁶. Recognizing this, the in-vitro model parameters are selected to address the study's objective while considering the clinical conditions. For example, the thrombotic occlusion study was conducted under more thrombogenic conditions in-vitro to simulate the situation where catheter patency is measurably compromised (which generally takes week or months in many patients). In contrast, the thrombus accumulation study was conducted under relatively less thrombogenic conditions to investigate the earlier stages of thrombus growth in the tip. Thus, this flexibility to enlist suitable in-vitro model conditions to address specific goals of a study represents an important strength of this model.

Regularly alternating the intraluminal blood flow direction in the Symetrex™ hemodialysis catheter was associated with lower thrombotic occlusion and lower thrombus accumulation in the tip region compared with conventional unidirectional flow as assessed in this in-vitro blood flow model.

As shown in FIG. 3, the thrombotic occlusion study showed that the Control group (unidirectional blood flow) was associated with a greater decrease in blood flow rate (48%±2%: mean±s.e.m) compared with the Test group with alternating flow direction (29%±6%: mean±s.e.m); Two-tailed paired t-test p-value<0.05, N=5. This finding implied that alternating blood flow direction regularly may help improve catheter patency and hence maintenance of hemodialysis blood flow.

Alternating flow direction in the context of the present invention means that a hemodialysis catheter having at least two extension tubes can be connected to a dialysis machine with one extension tube connects to the arterial patient connector, and the other extension tube connects to the venous patient connector of the dialysis machine in one dialysis treatment, and in a subsequent dialysis treatment, the extension tubes are connected to a patient connector other than the one that was connected to before. For example, when a first extension tube is connected to the arterial patient connector of a dialysis machine and the second extension tube is connected to the venous patient connector of a dialysis machine in one dialysis treatment, in the next dialysis treatment, the first extension tube is connected to the venous patient connector of a dialysis machine and the second extension tube is connected to the arterial patient connector of a dialysis machine. In other words, the extension tubes of a hemodialysis catheter are alternated between connecting to the arterial patient connector and the venous patient connector in consecutive dialysis treatments.

Connection indicators that can facilitate the recognition of connectivity of an extension tube of a hemodialysis catheter to a patient connector of a dialysis machine can be attached to a part of the hemodialysis catheter. Connection indicators can be attached to the hub, extension tubes, clamps, or connectors of the hemodialysis catheter.

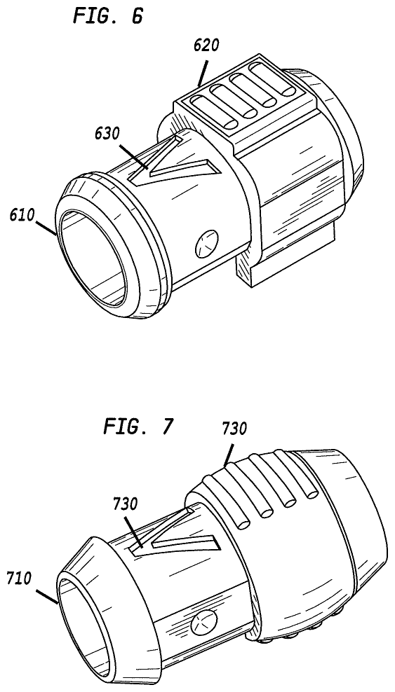

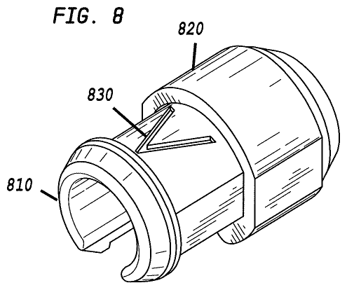

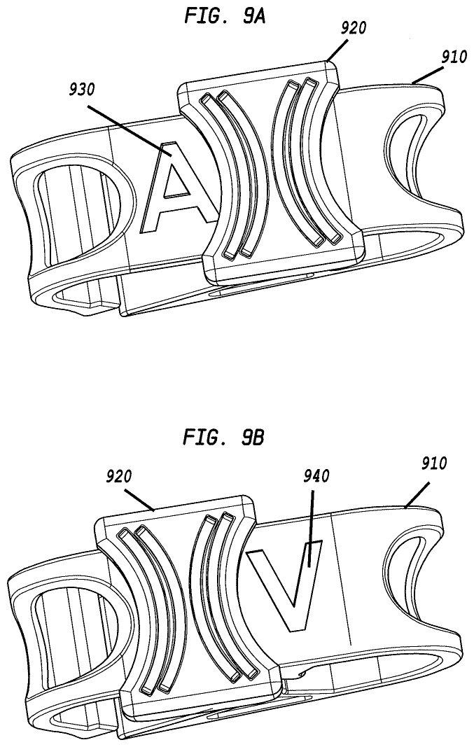

FIGS. 6-8 illustrate several embodiments of connection indicators that can be affixed to a hemodialysis catheter. In general, the connection indicator can be switched to indicate an extension tube is connected to the arterial patient connector or a venous patient connector of a dialysis machine. The connection indicator also can preferably be able to switch between two states (i.e., arterial and venous connection) or be moved from one extension tube to another extension tube. As non-limiting examples, embodiment shown in FIGS. 6-8 can be switched from indicating venous connection by the exposed letter V to indicating arterial connection by sliding a collar on the indicator to occlude the letter V and expose the letter A (not shown). The placement of the letter on the connection indicator can also be used to indicate the direction of blood flow. For example, the tip of the letter A and bottom of the letter V can be used as arrows to indicate blood flow direction.

In some of the embodiments, the switchable connection indicator comprises a barrel section 610 710 810, and a slidable sleeve 620 720 820, wherein the slidable sleeve can be slid along the barrel section between two positions, wherein a first position of the slidable sleeve on the barrel section indicate arterial connection, and a second position of the slidable sleeve on the barrel section indicate venous connection.

The slidable sleeve of the switchable connection indicator may be slid on the barrel section that can expose a first marking indicating arterial or venous connection, and occlude a second marking indicating arterial or venous connection 630 730 830. The markings may be letters, symbols, or colors (e.g., showing red color for arterial connection and blue color for venous connection).

The slidable sleeve is configured to remain at the first or the second position set by a user. For example, the slidable sleeve and the barrel section may comprise a depression and a protrusion. In one embodiment, a first depression is located on the exterior of the barrel section proximal to one end of the barrel section, and a second depression is located proximal to the other end of the barrel section. A protrusion is located on the interior surface of the slidable sleeve facing the barrel section, which can be accommodated by the depressions on the barrel section, preventing accidental movement of the slidable sleeve.

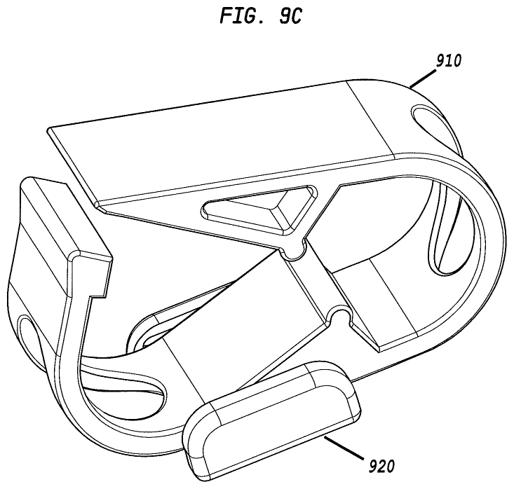

Connection indicators can be integrated with a clamp (FIGS. 9A-C). A clamp is typically placed along an extension tube of a dialysis catheter and can be used to temporarily restrict or stop flow through the extension tube. The construction of such clamp is well understood. Here a slide is shown to be integrated with one side 920 of the clamp 910. The slide 920 can be moved to show indicators for arterial connection 930 or venous connection 940, e.g., showing letter A or V (FIGS. 9A and 9B), or showing red color for arterial connection and blue color for venous connection. Integration of the flow indicators does not interfere with the function of the clamp.

The method of alternating flow of a hemodialysis catheter is preferably conducted with a catheter with low recirculation. Recirculation rate can be measure in vitro as described above. It is this advantageous to have visual or tactile indicator on a hemodialysis catheter to prevent accidentally using a hemodialysis catheter for the method of alternating flow.

Traditionally for hemodialysis catheters, the arterial flow path, which may include any one of extension tube, clamp, or connector, may be labeled red, either with the material or lettering. The venous flow path, which may include any one of extension tube, clamp, or connector, may be labeled blue, either with the material or lettering.

In one embodiment, the hemodialysis catheter with low recirculation may use the same color marking, either with the material or lettering, for any one of extension tube, clamp, or connector for the arterial and venous flow path. An exemplary implementation is to use the color green for any one of extension tube, clamp, or connector for the arterial and venous flow path. Colors other than red and blue, for example, brown, cyan, orange, violet, purple, black, etc., may be used for the same purpose.

It should be understood that many additional changes in the details, materials, steps and arrangements of parts, which have been herein described and illustrated in order to explain the nature of the present invention, may be made by those skilled in the art while still remaining within the principles and scope of the invention.

### REFERENCES

1. Sukavaneshvar S. Device thrombosis and pre-clinical blood flow models for assessing antithrombogenic efficacy of drug-device combinations. Adv Drug Deliv Rev. 2016 Aug. 3. pii: S0169-409X(16)30229-0. doi: 10.1016/j.addr.2016.07.009.
2. S. Sukavaneshvar. Assessment and Management of Vascular Implant Thrombogenecity (pgs57-78). In: Thrombus and Stroke. Informa Health Care (New York, NY), 2008
3. S. Sukavaneshvar. In-vitro assessment of device thrombosis and thromboembolism. Proceedings of Roadmap to Successful Development and Regulatory Approval of Medical Devices with Hemocompatible Coatings Workshop, Surfaces in Biomaterials Foundation. Scottsdale, A Z, August 2001.
4. M. Lotito, S. Sukavaneshvar, D. Olsen, J. Griggs, E. Jimenez, J. Bingham, L. Anglin, M. Langston, and G. Contreras. Thromboresistance Provided by a Heparin Coating on the Tal PALINDROMETM EMERALD Hemodialysis Catheter. 39th annual meeting of American Society of Nephrology, San Diego, November 2006
5. A. Dwyer, S. Sukavaneshvar, S. Nimkar, and G. Aronoff. Surface Heparinization of Hemodialysis Catheters Reduces Thrombus and Fibrin Sheath Formation. ASDIN 2006.
6. Smith R S, Zhang Z, Bouchard M, Li J, Lapp H S, Brotske G R, Lucchino D L, Weaver D, Roth L A, Coury A, Biggerstaff J, Sukavaneshvar S, Langer R, Loose C. Vascular catheters with a nonleaching poly-sulfobetaine surface modification reduce thrombus formation and microbial attachment. Sci Transl Med. 2012 Sep. 26; 4(153):153ra132.
7. Joseph Griggs, Ernesto Jimenez, Jodi Bingham, Sivaprasad Sukavaneshvar. Thrombosis and Thromboembolism Associated with Intravascular Catheter Biomaterials. World Biomaterials Congress, Amsterdam, N L, May 2008. P-Sat-M-708.

1. A hemodialysis catheter, comprising
a first and a second extension tubes;
a body comprising at least a first and second lumen;
a low recirculation distal tip;
a hub connecting the body and the first and second extension tubes;
a clamp on each of the first and second extension tubes;
a connector on each of the first and second extension tubes; and
a switchable connection indicator indicating connection to arterial or venous flow path,
wherein the first and second extension tubes are configured to be connected to a dialysis machine,
wherein the first lumen is in fluid communication with the first extension tube, and the second lumen is in fluid communication with the second extension tube,
wherein the low recirculation distal tip comprising a first and second distal openings, wherein the first distal opening is in fluid communication with the first lumen and the first extension tube, and the second distal opening is in fluid communication with the second lumen and the second extension tube.

2. The hemodialysis catheter of claim 1, wherein the switchable connection indicator comprising a barrel section, and a slidable sleeve, wherein the slidable sleeve can be slid along the barrel section between two positions, wherein a first position of the slidable sleeve on the barrel section indicate arterial connection, and a second position of the slidable sleeve on the barrel section indicate venous connection.

3. The hemodialysis catheter of claim 2, wherein sliding the slidable sleeve on the barrel section exposes a first marking indicating arterial or venous connection and occluding a second marking indicating arterial or venous connection.

4. The hemodialysis catheter of claim 2, wherein the slidable sleeve is configured to remain at the first or the second position set by a user.

5. The hemodialysis catheter of claim 2, wherein the slidable sleeve and the barrel section comprising corresponding structure for maintaining the slidable sleeve at first or the second position.

6. The hemodialysis catheter of claim 2, wherein the slidable sleeve and the barrel section comprising a depression and a protrusion.

7. The hemodialysis catheter of claim 2, wherein
a first depression is located on an exterior of the barrel section proximal to one end of the barrel section, and a second depression is located proximal to another end of the barrel section, and
a protrusion is located on an interior surface of the slidable sleeve facing the barrel section, which can be accommodated by the first and second depressions on the barrel section, preventing accidental movement of the slidable sleeve.

8. The hemodialysis catheter of claim 1, wherein the switchable connection indicator is a clamp comprising:
a slide, wherein the slide can be slid along a side of the clamp between two positions, wherein a first position of the slide indicates arterial connection, and a second position of the slide indicate venous connection.

9. The hemodialysis catheter of claim 8, wherein sliding the slide exposes a first marking indicating arterial or venous connection and occluding a second marking indicating arterial or venous connection.

10. The hemodialysis catheter of claim 8, wherein the slide is configured to remain at the first or the second position set by a user.
